(19) United States
(12) Patent Application Publication
(10) Pub. No.: US 2026/0279598 A1
(43) Pub. Date: Sep. 17, 2026

(54) FEDERATED SURVIVAL ANALYSIS WITH PRIVACY-PRESERVING AGGREGATION

(71) Applicants: Navid Seidi, Rolla, MO (US); Ardhendu Tripathy, College Park, MD (US); Sajal Das, Rolla, MO (US)

(72) Inventors: Navid Seidi, Rolla, MO (US); Ardhendu Tripathy, College Park, MD (US); Sajal Das, Rolla, MO (US)

(73) Assignee: The Curators of the University of Missouri, Columbia, MO (US)

(21) Appl. No.: 19/569,485

(22) Filed: Mar. 17, 2026

Related U.S. Application Data
(60) Provisional application No. 63/773,202, filed on Mar. 17, 2025.

(51) Int. Cl. G16H 50/80; G16H 40/20

(52) U.S. Cl. CPC G16H 50/80; G16H 40/20

(57) ABSTRACT

A method and systems for updating a federated survival model and generating personalized treatment recommendations. A plurality of local survival modeling nodes are each deployed at healthcare institutions. Each local survival modeling node trains a local survival model on institution-specific patient data. When the updates exceed a survival performance metric beyond a predetermined value, the local survival modeling nodes inject a calibrated noise into the local model update parameters to generate privacy-preserved model parameters to transmit to the federated learning server. The federated learning server generates feature availability vectors to aggregate the privacy-preserved model parameters into updated global model parameters. During the personalized treatment recommendation, the local survival modeling nodes integrate the updated global model parameters with the embedded feature representations of the patient's data. The local survival modeling node generates ranked treatment options for the patient based on a patient-specific survival prediction and local contextual factors.

### CROSS-REFERENCE TO RELATED APPLICATIONS

This application claims priority to U.S. Provisional Patent Application No. 63/773,202, filed Mar. 17, 2025, which is hereby incorporated by reference in its entirety.

### STATEMENT OF FEDERALLY SPONSORED RESEARCH OR DEVELOPMENT

This invention was made with government support under grant 2330769 awarded by the National Science Foundation. The government has certain rights in the invention.

### BACKGROUND

Healthcare providers across diverse geographic and institutional settings generate substantial volumes of patient data, yet this information remains distributed across independent systems without mechanisms for collaborative analysis. A physician at a rural hospital and a physician at a large urban medical center each maintain patient records reflecting their respective patient populations, treatment patterns, and outcome histories. While both physicians seek to provide personalized care recommendations based on the broadest possible evidence base, regulatory constraints and privacy requirements prevent direct sharing of patient-level data between institutions. Federated learning approaches have emerged as alternatives to centralized data aggregation, enabling collaborative model training without transferring raw patient records. However, healthcare institutions differ in the clinical variables they collect, the diagnostic equipment they employ, and the patient populations they serve, resulting in heterogeneous data that presents challenges for conventional collaborative learning frameworks.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In one aspect, a method for updating a federated survival model across a plurality of healthcare institutions includes receiving, at a federated learning server, privacy-preserved model parameters from a plurality of local survival modeling nodes. Each local survival modeling node is deployed at a respective healthcare institution and is configured to train a local survival model to generate local model update parameters using embedded feature representations of institution-specific patient data and censored outcome data, apply differential privacy noise to the local model update parameters to generate the privacy-preserved model parameters, and transmit the privacy-preserved model parameters to the federated learning server. The method further includes determining, by the federated learning server, based on the received model parameters and clustering, by the federated learning server, the healthcare institutions into groups based on similarity of the feature presence patterns. The method also includes computing, within each cluster, component-wise weighted parameter averages using weights derived from institutional sample size and disease-severity metrics to generate updated global model parameters and distributing the updated global model parameters to the plurality of local survival modeling nodes.

In another aspect, a method for generating personalized treatment recommendations using a federated survival model includes receiving, at a local survival modeling node deployed at a healthcare institution, updated global model parameters from a federated learning server. The updated global model parameters are generated by clustering a plurality of healthcare institutions into groups based on feature presence patterns, and computing, within each cluster, component-wise weighted parameter averages using weights derived from institutional sample size and disease-severity metrics. The method further includes receiving, at the local survival modeling node, patient data for a patient and transforming the patient data into embedded feature representations. The method also includes generating, by a personalized inference engine at the local survival modeling node, a patient-specific survival prediction by integrating the updated global model parameters with the embedded feature representations and processing, by a contextual constraint processor, local contextual factors for the patient, the local contextual factors comprising at least one of cost constraints, insurance coverage, or treatment availability. The method additionally includes generating, by a treatment recommendation module, ranked treatment options for the patient based on the patient-specific survival prediction and the local contextual factors.

In yet another aspect, a decentralized federated learning system includes a plurality of local survival modeling nodes. Each local survival modeling node is deployed at a respective healthcare institution and includes a local survival processor and a local survival memory coupled to the local survival processor. The local survival memory stores processor-executable instructions that, when executed, configure the local survival processor for training a local survival model on institution-specific patient data, computing local model update parameters based on censored outcome data, injecting a calibrated noise parameter into the local model update parameters to generate privacy-preserved model parameters, and transmitting the privacy-preserved model parameters to a federated learning server. The federated learning server includes a federated learning processor and a federated learning memory coupled to the federated learning processor. The federated learning memory stores processor-executable instructions that, when executed, configure the federated learning processor for receiving the privacy-preserved model parameters from the plurality of local survival modeling nodes, generating feature availability vectors based on feature presence at each healthcare institution, clustering the healthcare institutions into groups based on similarity of the feature availability vectors, executing a severity-weighted aggregation engine configured to compute, within each cluster, component-wise weighted parameter averages using weights derived from institutional sample size and disease-severity metrics to generate updated global model parameters, and distributing the updated global model parameters to the plurality of local survival modeling nodes.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a block diagram of a federated learning system architecture, according to an embodiment.

FIG. 2 illustrates a swim lane diagram of a local survival modeling workflow, according to an embodiment.

FIG. 3 illustrates a swim lane diagram of a federated learning server workflow, according to an embodiment.

FIG. 4 illustrates a flowchart of a local model training process for updating a federated survival model, according to an embodiment.

FIG. 5 illustrates a personalized inference and treatment recommendation pipeline, according to an embodiment.

### DETAILED DESCRIPTION

The features and other details of the concepts, systems, and techniques sought to be protected herein will now be more particularly described. It will be understood that any specific embodiments described herein are shown by way of illustration and not as limitations of the disclosure and the concepts described herein. Features of the subject matter described herein can be employed in various embodiments without departing from the scope of the concepts sought to be protected.

Modern healthcare systems generate considerable volumes of clinical, genomic, imaging, and outcome data across geographically distributed institutions. Regulatory constraints, institutional governance policies, and patient privacy requirements prevent centralized aggregation of patient-level data into unified repositories. Healthcare institutions operate as data silos, where each institution maintains patient records independently without systematic mechanisms for sharing raw data with external entities. This fragmentation limits the statistical power available for training predictive models, particularly survival models that benefit from large and diverse patient populations. Rural hospitals and under-resourced healthcare centers face additional challenges, as these institutions lack access to broad population-level outcome data that would otherwise inform treatment decisions.

Privacy concerns present a substantial barrier to collaborative healthcare analytics. Regulations such as the Health Insurance Portability and Accountability Act (HIPAA) impose strict requirements on the handling and transmission of protected health information. Traditional approaches to multi-institutional research require complex data use agreements, de-identification procedures, and centralized data warehousing arrangements that introduce security risks and governance overhead. These barriers delay or prevent collaborative research initiatives that would otherwise improve patient outcomes through access to larger and more representative datasets.

Data heterogeneity across healthcare institutions presents additional technical challenges for collaborative model training. Different institutions collect different sets of clinical variables based on local practices, available diagnostic equipment, patient populations, and electronic health record configurations. Feature spaces vary across institutions, with some institutions capturing genomic markers while others focus on imaging-derived features or laboratory measurements. Patient populations also differ in demographic composition, disease severity distributions, and treatment patterns. These variations result in non-independent and non-identically distributed (non-IID) data that degrades the performance of conventional federated learning approaches designed for aligned feature spaces.

Survival analysis presents specialized conditions that conventional federated learning frameworks do not address. Survival models operate on censored outcome data, where the event of interest (such as disease progression or mortality) has not occurred for some patients at the time of analysis. The statistical structure of survival models, including hazard functions and time-to-event distributions, requires aggregation methods tailored to survival-specific parameters rather than generic model weights. Existing clinical decision support systems remain locally constrained, relying on institution-specific data without mechanisms for incorporating knowledge learned from broader patient populations.

The present disclosure describes a decentralized federated survival analysis and personalized treatment recommendation system that addresses these challenges. The system enables multiple healthcare institutions to collaboratively train survival models without sharing patient-level data. The architecture comprises local survival modeling nodes deployed at each participating healthcare institution and a federated learning server that coordinates model aggregation across institutions. Each local survival modeling node trains survival models on institution-specific patient data and transmits privacy-preserved model parameters to the federated learning server. The local survival modeling nodes retain local patient data local at each institution to ensure patient privacy. The federated learning server clusters institutions based on feature presence patterns to address heterogeneous feature spaces, computes severity-weighted parameter averages within each cluster, and distributes updated global model parameters back to the local survival modeling nodes. A personalized inference engine at each local survival modeling node integrates the updated global model parameters with local contextual factors to generate patient-specific treatment recommendations. This architecture preserves patient privacy by ensuring that raw patient data remains at the originating healthcare institution while enabling collaborative learning from multi-institutional datasets.

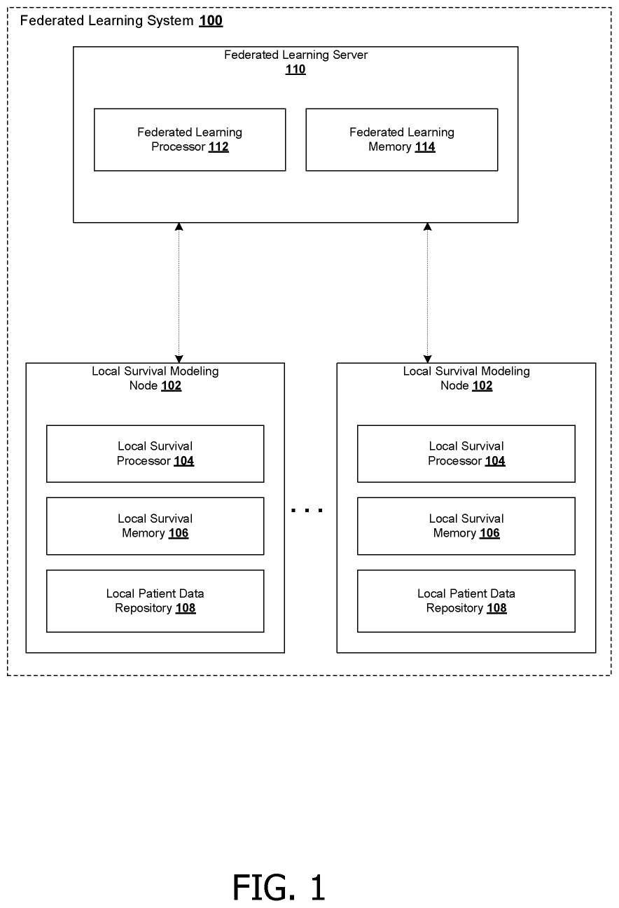

Referring to FIG. 1, a federated learning system 100 comprises a three-layer architecture that enables collaborative survival model training across distributed healthcare institutions. The three layers include an institution nodes layer, a global coordination layer, and an inference layer. The institution nodes layer comprises a plurality of local survival modeling nodes 102, each deployed at a respective healthcare institution. The global coordination layer comprises a federated learning server 110 that coordinates model aggregation across the plurality of local survival modeling nodes 102. The inference layer operates at each local survival modeling node 102 to generate patient-specific predictions and treatment recommendations using aggregated model parameters.

Each local survival modeling node 102 comprises a local survival processor 104, a local survival memory 106 coupled to the local survival processor 104, and a local patient data repository 108. The local survival memory 106 stores instructions that enable the local survival modeling node 102 to perform local model training, privacy protection, and inference operations. In some embodiments, multiple local survival modeling nodes 102 are deployed at a single healthcare institution to support different clinical departments, research groups, or patient populations within that institution.

The local patient data repository 108 stores institution-specific patient data including electronic health records, clinical registry data, genomic data, imaging-derived features, and treatment outcome records. The local patient data repository 108 maintains patient data locally at the healthcare institution, ensuring that raw patient data does not leave the originating institution during the federated learning process. Electronic health records comprise structured and semi-structured patient data including demographic information, medical history, diagnosis codes, procedure codes, medication records, vital signs, and clinical notes. Clinical registry data comprises standardized outcome data collected through disease-specific registries such as cancer registries, transplant registries, and chronic disease registries that track patient outcomes over extended time periods. Genomic data comprises genetic sequencing results, gene expression profiles, mutation status indicators, and pharmacogenomic markers that characterize the molecular characteristics of patient conditions. Imaging-derived features comprise quantitative measurements extracted from medical imaging studies including radiographic images, computed tomography scans, magnetic resonance imaging studies, and pathology slides. Treatment outcome records comprise time-to-event data including survival times, disease progression dates, treatment response assessments, and censoring indicators that characterize whether the event of interest has occurred for each patient at the time of analysis.

With continued reference to FIG. 1, the federated learning server 110 comprises a federated learning processor 112 and a federated learning memory 114 coupled to the federated learning processor 112. The federated learning memory 114 stores instructions that enable the federated learning server 110 to coordinate model aggregation across the distributed network of local survival modeling nodes 102. The federated learning server 110 operates as a coordination hub that facilitates collaborative learning without accessing raw patient data from any participating healthcare institution. While FIG. 1 illustrates the federated learning server 110 as a single server, the decentralized network includes, in some embodiments, multiple federated learning servers 110 in communication with each other to coordinate the learning process across geographically distributed regions or organizational boundaries.

As further shown in FIG. 1, bidirectional connections between the local survival modeling nodes 102 and the federated learning server 110 represent the network between the local survival modeling nodes 102 and the federated learning server 110. The network facilitates the exchange of privacy-preserved model parameters and updated global model parameters. Each local survival modeling node 102 transmits privacy-preserved model parameters to the federated learning server 110 after local model training while retaining raw patient data in the local patient data repository 108. The federated learning server 110 processes the received parameters, performs clustering and aggregation operations, and distributes updated global model parameters back to the local survival modeling nodes 102. This bidirectional exchange through the network enables each local survival modeling node 102 to benefit from knowledge learned across all participating healthcare institutions while maintaining patient data residency at the originating institution.

The federated learning system 100 supports alternative federated topologies that replace the centralized coordination architecture with decentralized network configurations. In some embodiments, the federated learning server 110 is replaced by a peer-to-peer topology wherein healthcare institutions form a peer-to-peer network and aggregation occurs dynamically without a central coordinator. The peer-to-peer topology enables decentralized consensus-based parameter updates that distribute the coordination responsibilities across the participating healthcare institutions rather than concentrating coordination at a single federated learning server. Aggregation in the peer-to-peer topology occurs through distributed consensus protocols that enable healthcare institutions to converge on shared model parameters without centralized coordination. The peer-to-peer topology eliminates the single point of coordination that the federated learning server represents in the centralized architecture. Thus, healthcare institutions in the peer-to-peer network may establish direct communication channels with peer institutions based on network connectivity, institutional relationships, or feature presence similarity. In some embodiments, the federated learning system 100 incorporates game-theoretic peer selection for alternative federated topologies to optimize the selection of peer institutions for parameter exchange. Game-theoretic peer selection models the peer selection process to enable healthcare institutions to select peers that maximize the benefit received from the federated learning process while accounting for the costs of participation.

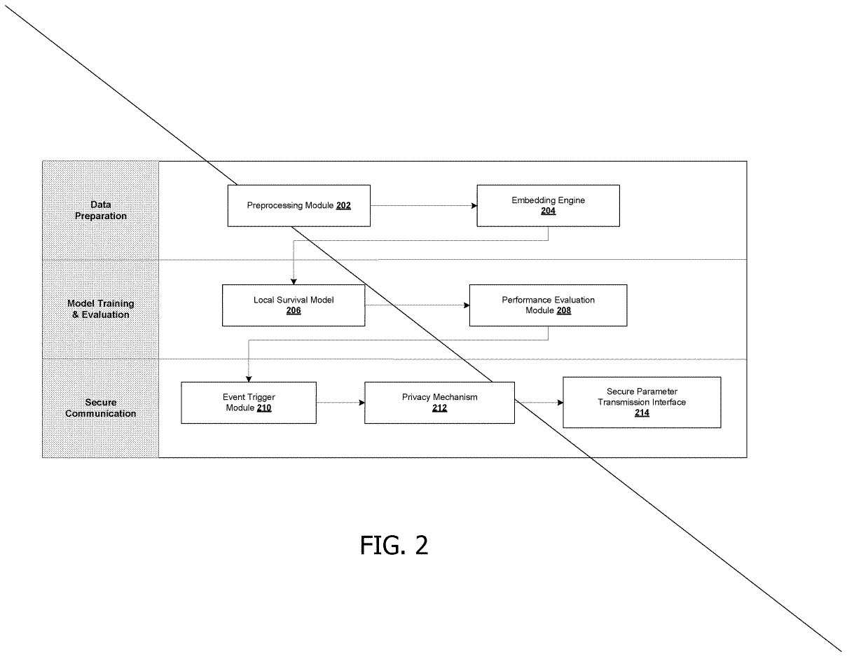

Referring to FIG. 2, the local survival modeling workflow at an individual healthcare institution operates through four functional lanes: a data preparation lane, a model training and evaluation lane, a secure communication lane, and a personalized inference lane. The data preparation lane, the model training and evaluation lane, and the secure communication lane collectively enable each local survival modeling node 102 to train survival models on institution-specific patient data, evaluate model performance, and transmit privacy-preserved model parameters to the federated learning server 110. The personalized inference lane, described in further detail below, generates patient-specific treatment recommendations using aggregated model parameters received from the federated learning server 110. The local survival modeling workflow may be executed by a local survival modeling node 102 at a healthcare institution.

The data preparation lane further includes a pre-processing module 202 that performs data pre-processing locally at each participating healthcare institution prior to model training to prepare heterogeneous patient data for survival modeling. The pre-processing module 202 executes pre-processing operations including data normalization and standardization that transform numerical variables to common scales, missing value handling or imputation that addresses incomplete patient records, feature encoding for categorical variables that converts non-numerical attributes to numerical representations, and time-to-event formatting that structures outcome data for survival analysis. The pre-processing module 202 also performs removal of identifiers or protected health information when the institutional data governance policies require such removal before model training. The pre-processing module 202 executes these pre-processing steps locally within each healthcare institution so that raw patient data does not leave the originating site during the federated learning process. The pre-processing module 202 provides the preprocessed patient data to the embedding engine 204 for transformation into high-dimensional feature vectors.

The data preparation lane includes an embedding engine 204 that transforms institution-specific patient data from the local patient data repository 108 into high-dimensional feature vectors suitable for survival modeling. The embedding engine 204 receives heterogeneous clinical attributes including demographic variables, clinical measurements, treatment variables, genomic indicators, laboratory values, and other contextual health features. The embedding engine 204 applies feature normalization, encoding of categorical variables, and learned representations to convert the heterogeneous patient data into structured numerical feature vectors. In one embodiment, the embedding engine 204 transforms patient data into high-dimensional vector spaces using the formula:

$${E}_{k} = f ( {X}_{k} , {θ}_{e} )$$

where Eₓ is the embedding for patient k, Xₖ is the raw patient data, and θₑ are learned embedding parameters. In some embodiments, the embedding engine 204 incorporates multimodal data sources including genomic data, imaging-derived features, laboratory measurements, wearable device data, and insurance claims data into a unified survival-optimized embedding space. The resulting high-dimensional feature vectors represent each patient in a structured feature space that the local survival model 206 processes during training.

In one embodiment, the embedding engine 204 uses learned representations derived from machine learning models for transforming patient data into feature vectors. The machine learning models learn embedding parameters from training data that optimize the representation of patient characteristics for downstream survival modeling tasks. In some embodiments, the embedding engine 204 uses transformer-based survival embeddings for feature representation in some embodiments. The transformer architecture enables the embedding engine to integrate information from multiple data modalities and generate unified patient representations that capture complex interactions among clinical, genomic, imaging, and behavioral features relevant to survival outcomes.

With continued reference to FIG. 2, the model training and evaluation lane includes a local survival model 206 and a performance evaluation module 208. The local survival model 206 trains on the embedded feature representations generated by the embedding engine 204 and computes local model update parameters using censored outcome data from the local patient data repository 108. Censored outcome data includes time-to-event information where the event of interest, such as disease progression, treatment response, or mortality, has not occurred for some patients at the time of analysis. Censored observations occur when patients are lost to follow-up, when the study observation period ends before the event occurs, or when patients experience competing events that preclude observation of the event of interest. The local survival model processes censored outcome data using likelihood functions that account for both observed events and censored observations. For patients with observed events, the likelihood function incorporates the exact event time and the hazard at that time. For patients with censored observations, the likelihood function incorporates the survival probability up to the censoring time without assuming knowledge of when the event would have occurred. Censored outcome data handling addresses the statistical structure of survival analysis where the event of interest has not occurred for some patients at the time of analysis. This censored outcome data handling enables the local survival model to extract information from all available patient records, including patients who have not yet experienced the event of interest, maximizing the statistical power available for model training at each healthcare institution.

The local survival model 206 computes model parameters including hazard coefficients and baseline hazard estimates that characterize the relationship between patient features and survival outcomes. In an embodiment, the local survival model 206 performs federated survival analysis by computing local model update parameters according to the formula:

$${θ}_{local , i} = arg {max}_{θ} L ( θ \vert {E}_{i} , {Y}_{i} )$$

where θ_{local,i} are the model parameters for institution i, Eᵢ are embedded patient features, and Yᵢ are survival outcomes. In some embodiments, the local survival model 206 comprises a Cox proportional hazards model that estimates hazard ratios for each feature in the embedded feature representations. The Cox proportional hazards model models the hazard function as a product of a baseline hazard function and an exponential function of the linear combination of patient features. The Cox proportional hazards model computes hazard coefficients that quantify the effect of each feature on the instantaneous risk of the event of interest occurring. In an embodiment, the Cox proportional hazards model estimates the hazard coefficients. The model does so by maximizing a partial likelihood function that accounts for the relative ordering of event times among patients without requiring specification of the baseline hazard function. For each patient with an observed event, the partial likelihood function computes the probability that the event occurred for that patient given the set of patients at risk at the event time. The Cox proportional hazards model iteratively updates the hazard coefficients using gradient-based optimization methods until convergence criteria are satisfied. The resulting hazard coefficients represent the local model update parameters that the privacy module 212 processes before transmission to the federated learning server 110.

While the above-described example uses a Cox proportional model, the federated learning system 100 is model agnostic and may be implemented using time-to-event modeling approaches. In other embodiments, the local survival model 206 comprises neural survival models or other survival models adapted for the specific clinical application. Neural survival models apply deep learning architectures to model the relationship between patient features and survival outcomes, enabling the local survival model to capture complex nonlinear relationships and feature interactions that the proportional hazards assumption of Cox models does not accommodate. The neural survival model generates local model update parameters comprising neural network weights that the privacy module 212 processes before transmission to the federated learning server.

The performance evaluation module 208 evaluates model performance using a survival analysis metric after the local survival model 206 computes the local model update parameters. In some embodiments, the survival analysis metric comprises a concordance index that measures the proportion of patient pairs for which the local survival model 206 correctly ranks survival times based on predicted risk scores. In other embodiments, the survival analysis metric comprises a log-likelihood improvement that measures the change in model fit between successive training iterations. In still other embodiments, the survival analysis metric comprises a predictive performance measure. The performance evaluation module 208 computes the survival analysis metric based on the local model update parameters and compares the computed metric against historical performance values to determine whether the local survival model 206 has achieved a performance improvement.

As further shown in FIG. 2, an event trigger module 210 in the secure communication lane determines when performance thresholds are met to initiate transmission of model parameters to the federated learning server 110. The event trigger module 210 receives the survival analysis metric from the performance evaluation module 208 and compares the metric against a predefined performance threshold. Transmitting the privacy-preserved model parameters occurs in response to the survival analysis metric exceeding the predefined performance threshold. In some embodiments, the predefined performance threshold comprises a concordance index increase exceeding a predefined margin such as 0.01 to 0.02 between successive evaluation periods. Thus, the event trigger module 210 reduces communication overhead by transmitting model updates to the federated learning server 110 when the local survival model 206 achieves a performance improvement rather than transmitting updates at fixed intervals regardless of model progress. This approach conserves network bandwidth and computational resources at the federated learning server by filtering out model updates that do not contribute meaningful improvements to the aggregated global model parameters.

In some embodiments, the event trigger module 210 initiates transmission in response to additional adaptive triggers including availability of new patient outcome data, completion of a local training cycle, a periodic synchronization interval, or detection of a change in data distribution at the healthcare institution. In this way, the event trigger module 210 may incorporate adaptive communication policies for determining when to transmit model updates beyond the performance threshold mechanism. These adaptive communication policies enable the federated learning system 100 to respond to various conditions at participating healthcare institutions that warrant model parameter transmission. The event trigger module 210 may evaluate multiple adaptive triggers concurrently and initiates transmission when any trigger condition is satisfied.

The adaptive communication policies utilized by the event trigger module 210 may include a response to availability of new patient outcome data at a healthcare institution. Healthcare institutions continuously collect patient outcome data as patients complete treatment courses, experience disease progression events, or reach follow-up milestones. When new patient outcome data becomes available, the local survival model incorporates this data during subsequent training iterations, and the resulting model updates reflect the additional survival information. Alternatively, or additionally, the adaptive communication policies may respond to a periodic synchronization interval. The periodic synchronization interval defines a time-based schedule for transmitting model updates regardless of performance improvements or other event-based triggers. Healthcare institutions configure the periodic synchronization interval based on operational requirements, network availability, and coordination policies established for the federated learning system. In yet another embodiment, the adaptive communication policies may respond to detection of a change in data distribution at a healthcare institution. Data distribution changes occur when the patient population at a healthcare institution shifts in demographic composition, disease severity distribution, treatment patterns, or feature availability. The event trigger module 210 monitors characteristics of the local patient data and detects changes in data distribution by comparing current data statistics against historical baselines. When the event trigger module detects a change in data distribution that exceeds a predefined threshold, the event trigger module initiates transmission of privacy-preserved model parameters to the federated learning server.

Referring again to FIG. 2, a privacy module 212 in the secure communication lane injects a calibrated noise parameter into the local model update parameters to generate privacy-preserved model parameters before transmission to the federated learning server 110. The privacy module 212 applies differential privacy techniques that add noise calibrated according to differential privacy principles to the local model update parameters. In some embodiments, the privacy module 212 performs privacy-preserving model aggregation according to the formula:

$${θ}_{g l obal} = \frac{1}{N} {\sum }_{i = 1}^{N} {w}_{i} \cdot {θ}_{local , i} + η$$

where wⁱ are institution-specific weights and η is noise added for differential privacy. The calibrated noise parameter prevents reconstruction of individual patient records from the transmitted model parameters while preserving statistical characteristics of the local model update parameters that enable meaningful aggregation at the federated learning server 110. Because aggregation occurs across multiple healthcare institutions, the noise does not materially alter the predictive properties of the aggregated global model parameters. The privacy module 212 ensures that the federated learning system 100 satisfies differential privacy guarantees that protect patient privacy during collaborative model training.

With continued reference to FIG. 2, a secure parameter transmission interface 214 transmits the privacy-preserved model parameters to the federated learning server 110. The secure parameter transmission interface 214 establishes a secure communication channel between the local survival modeling node 102 and the federated learning server 110 for transmitting the privacy-preserved model parameters. The secure parameter transmission interface 214 transmits model parameters rather than raw patient data, ensuring that patient-level information remains at the originating healthcare institution throughout the federated learning process. In some embodiments, the secure parameter transmission interface 214 applies one or more security mechanisms to the privacy-preserved model parameters before transmission. The security mechanism may include encryption or an access control policy. Encryption protects the privacy-preserved model parameters during transit over the network connecting the local survival modeling node 102 to the federated learning server 110. The access control policy restricts which entities receive and process the privacy-preserved model parameters at the federated learning server 110. In one embodiment, the access control policy implements role-based access control that assigns permissions based on the role of each entity within the federated learning system, while in other embodiments, the access control policies implement attribute-based access control that evaluates attributes of the requesting entity, the requested resource, and the environmental context to determine access permissions.

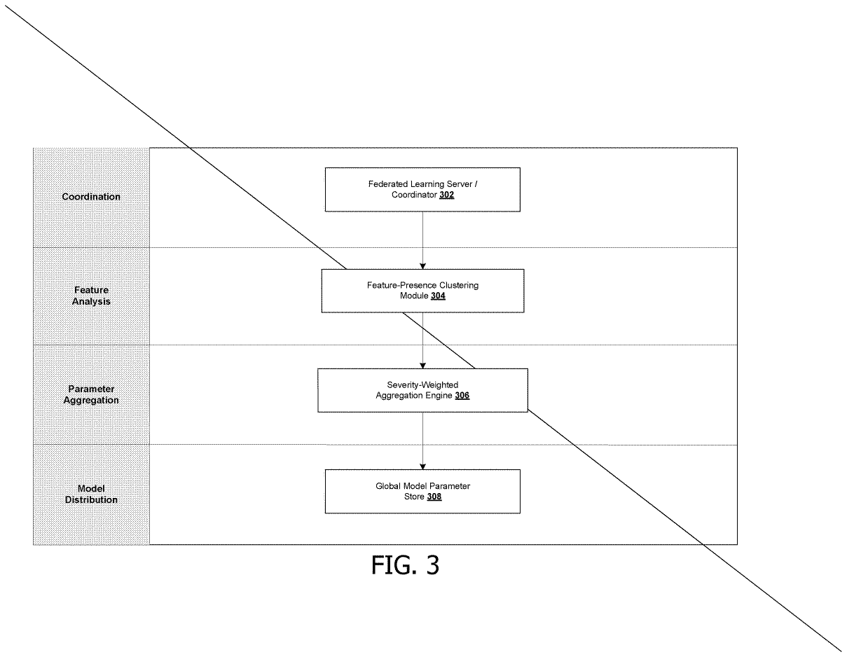

Referring to FIG. 3, the federated learning server workflow operates through four functional lanes: a coordination lane, a feature analysis lane, a parameter aggregation lane, and a model distribution lane. These four lanes collectively enable the federated learning server 110 to receive privacy-preserved model parameters from the plurality of local survival modeling nodes 102, cluster healthcare institutions based on feature presence patterns, compute severity-weighted parameter averages, and distribute updated global model parameters back to the local survival modeling nodes 102.

The coordination lane includes a federated learning coordinator 302 that receives the privacy-preserved model parameters from the plurality of local survival modeling nodes 102. The federated learning coordinator 302 operates as the central coordination point for the federated learning system 100, receiving encrypted model updates from each participating healthcare institution without accessing raw patient data. As described above with reference to FIG. 2, each local survival modeling node 102 transmits privacy-preserved model parameters through the secure parameter transmission interface 214 after the privacy module 212 injects calibrated noise into the local model update parameters. The federated learning coordinator 302 collects these privacy-preserved model parameters from all participating local survival modeling nodes 102 and prepares the parameters for subsequent clustering and aggregation operations.

With continued reference to FIG. 3, the feature analysis lane includes a feature-presence clustering module 304 that generates feature availability vectors based on feature presence at each healthcare institution. Different healthcare institutions collect different sets of clinical variables based on local practices, available diagnostic equipment, patient populations, and electronic health record configurations. The feature-presence clustering module 304 addresses this heterogeneity by analyzing which features are present in the model parameters received from each local survival modeling node 102. For each healthcare institution, the feature-presence clustering module 304 generates an aggregated vector embedding based on the feature set available at that institution. The aggregated vector embedding represents the feature presence pattern for the healthcare institution as a binary or weighted vector indicating which features the institution captures in its patient data. Thus, the feature-presence clustering module 304 enables to the federated learning system 100 to address data heterogeneity across institutions.

The feature-presence clustering module 304 clusters the healthcare institutions into groups based on similarity of the feature availability vectors. Healthcare institutions with similar feature presence patterns are grouped together to enable meaningful parameter aggregation over shared feature subsets. In some embodiments, the feature-presence clustering module 304 applies KMeans clustering to the feature presence patterns to partition the healthcare institutions into clusters. In one embodiment, the KMeans clustering algorithm minimizes the sum of squared distances between each feature availability vector and the mean feature pattern of the assigned cluster according to the formula:

$${min}_{{C}_{1} , {C}_{2} , … , {C}_{c}} {\sum }_{i = 1}^{C} {\sum }_{c ∈ {C}_{i}} {( {h}_{c} - {μ}_{i} )}^{2}$$

where Cᵢ is the set of centers in cluster i, c is the number of clusters, h_{c} is the aggregated vector embedding for center c, and μᵢ is the mean bit pattern of cluster i. This clustering approach ensures that parameter aggregation occurs among healthcare institutions that share common features, avoiding the degradation in model performance that would result from aggregating parameters corresponding to misaligned feature spaces.

As further shown in FIG. 3, the parameter aggregation lane includes a severity-weighted aggregation engine 306 that computes, within each cluster, component-wise weighted parameter averages using weights derived from institutional sample size and disease-severity metrics to generate updated global model parameters. The severity-weighted aggregation engine 306 performs aggregation over shared feature subsets within each cluster, computing weighted averages of model parameters corresponding to features that are common across all healthcare institutions in the cluster. For features that are present at some institutions but absent at others within a cluster, the severity-weighted aggregation engine 306 aggregates parameters from the subset of institutions that capture those features. Thus, the severity-weighted aggregation further addresses data heterogeneity across healthcare institutions by identifying shared feature subsets. The severity-weighted aggregation engine 306 computes weighted averages of beta coefficients for common features within each cluster using the formula:

$${β}_{global , i} = \frac{{\sum }_{c ∈ {C}_{i}} {r}_{c} \cdot {β}_{local , c} ( {F}_{common , i} )}{{\sum }_{c ∈ {c}_{i}} {r}_{c}}$$

where β_{global,i} represents the aggregated global beta coefficients for cluster i, Cᵢ represents the set of healthcare institutions in cluster i, r_{c} represents the number of rows in the dataset of center c (corresponding to the institutional sample size), β_{local,c} represents the local beta coefficients from center c, and F_{common,i} represents the common features in cluster i. This weighting scheme ensures that healthcare institutions with larger patient populations and more severe disease distributions contribute proportionally more to the aggregated global model parameters, improving generalization in survival contexts where sample size and disease severity affect parameter reliability.

The severity-weighted aggregation engine 306 incorporates trust or reputation weights in addition to sample size and severity metrics for parameter aggregation in some embodiments. Trust or reputation weights quantify the reliability or standing of each healthcare institution within the federated learning network. The severity-weighted aggregation engine combines the trust or reputation weights with the sample size weights and disease-severity weights to compute composite aggregation weights for each healthcare institution. This multi-factor weighting approach enables the federated learning system 100 to account for differences in data quality and institutional reliability across participating healthcare institutions while maintaining the sample size and severity weighting that improves generalization in survival contexts.

With continued reference to FIG. 3, the model distribution lane includes a global model parameter store 308 that stores the updated global model parameters generated by the severity-weighted aggregation engine 306. The global model parameter store 308 maintains the aggregated beta coefficients and other model parameters for each cluster, enabling retrieval and distribution of the updated global model parameters to the local survival modeling nodes 102. After the severity-weighted aggregation engine 306 completes the aggregation process, the global model parameter store 308 distributes the updated global model parameters to the plurality of local survival modeling nodes 102. Each local survival modeling node 102 receives the updated global model parameters corresponding to the cluster to which the healthcare institution belongs, enabling the local survival model 206 to incorporate knowledge learned from other healthcare institutions with similar feature presence patterns. This distribution completes the federated learning cycle and prepares each local survival modeling node 102 for subsequent local training iterations and personalized inference operations.

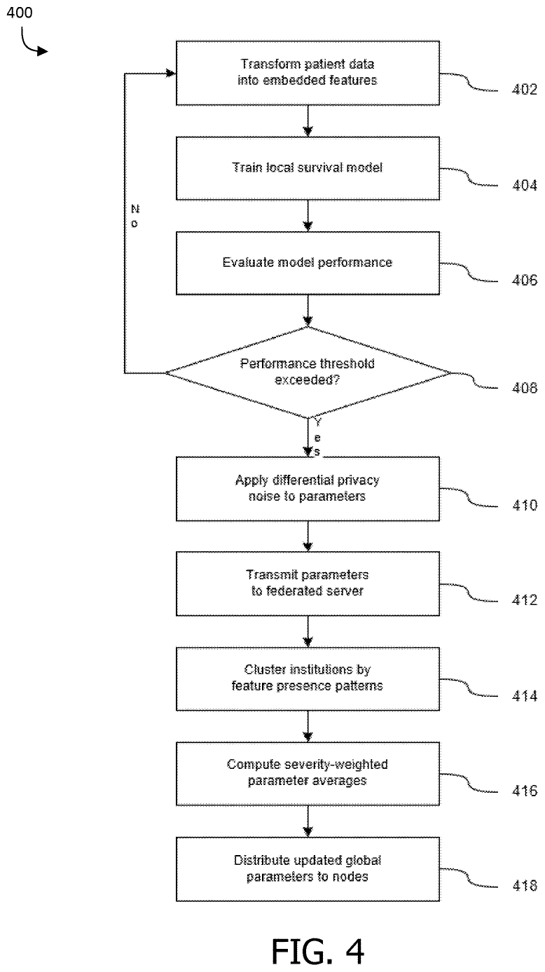

Referring to FIG. 4, a local model training process 400 for updating a federated survival model across a plurality of healthcare institutions comprises a sequence of steps performed at the local survival modeling nodes 102 and the federated learning server 110. The local model training process 400 enables collaborative survival model training while preserving patient privacy through differential privacy mechanisms and event-triggered communication. The local model training process 400 begins with local data transformation and model training operations at each participating healthcare institution and proceeds through privacy-preserving parameter transmission, federated clustering and aggregation, and distribution of updated global model parameters.

At a step 402 of the local model training process 400, the embedding engine 204 transforms patient data from the local patient data repository 108 into embedded feature representations. As described above with reference to FIG. 2, the embedding engine 204 receives heterogeneous clinical attributes including demographic variables, clinical measurements, treatment variables, genomic indicators, and laboratory values. The embedding engine 204 applies feature normalization and encoding of categorical variables to convert the heterogeneous patient data into high-dimensional feature vectors. At a step 404, the local survival model 206 trains using the embedded feature representations and censored outcome data from the local patient data repository 108. The local survival model 206 computes local model update parameters including hazard coefficients that characterize the relationship between patient features and survival outcomes.

Continuing with the local model training process 400, at a step 406, the performance evaluation module 208 evaluates model performance using a survival analysis metric. The performance evaluation module 208 computes the survival analysis metric based on the local model update parameters generated at step 404 and compares the computed metric against historical performance values to quantify the performance improvement achieved during the current training iteration. At a step 408, the event trigger module 210 determines whether a performance threshold is exceeded. The event trigger module 210 compares the survival analysis metric computed at step 406 against a predefined performance threshold, as described above. In some embodiments, the event trigger module 210 evaluates one or more adaptive communication policies to determine whether to transmit model updates. For example, as described above, the adaptive communication policy may include triggers for arrival of ne patient outcome data, measurable improvements to local model performance, and/or detectable shifts in local data distributions. If the survival analysis metric does not exceed the predefined performance threshold or the trigger of an adaptive communication policy, the local model training process 400 continues monitoring and returns to step 404 for additional local training iterations. If the survival analysis metric exceeds the predefined performance threshold or meets the trigger of an adaptive communication policy, the local model training process 400 proceeds to step 410 to initiate the privacy-preserving parameter transmission process.

The privacy module 212 applies differential privacy noise to the local model update parameters at a step 410 of the local model training process 400. As described above with reference to FIG. 2, the privacy module 212 injects a calibrated noise parameter into the local model update parameters to generate privacy-preserved model parameters. At a step 412, the secure parameter transmission interface 214 transmits the privacy-preserved model parameters to the federated learning server 110. The secure parameter transmission interface 214 establishes a secure communication channel and transmits model parameters rather than raw patient data, ensuring that patient-level information remains at the originating healthcare institution. The federated learning server 110 receives the privacy-preserved model parameters from the plurality of local survival modeling nodes 102 through the federated learning coordinator 302.

Continuing with the local model training process 400 at a step 414, the feature-presence clustering module 304 clusters the healthcare institutions into groups based on feature presence patterns. The federated learning server 110 determines feature presence patterns based on the received model parameters by analyzing which features are present in the model parameters received from each local survival modeling node 102. The feature-presence clustering module 304 generates feature availability vectors for each healthcare institution and clusters the healthcare institutions into groups based on similarity of the feature presence patterns. At a step 416, the severity-weighted aggregation engine 306 computes, within each cluster, component-wise weighted parameter averages using weights derived from institutional sample size and disease-severity metrics to generate updated global model parameters. Computing component-wise weighted parameter averages comprises aggregating parameters corresponding to shared features within each cluster. As described above with reference to FIG. 3, the severity-weighted aggregation engine 306 performs aggregation over shared feature subsets, computing weighted averages of model parameters corresponding to features that are common across all healthcare institutions in the cluster.

The global model parameter store 308 distributes the updated global model parameters to the plurality of local survival modeling nodes 102 at step 418 of the local model training process 400. Each local survival modeling node 102 receives the updated global model parameters corresponding to the cluster to which the healthcare institution belongs. The distribution of updated global model parameters completes the federated learning cycle described by local model training process 400 and enables each local survival modeling node 102 to incorporate knowledge learned from other healthcare institutions with similar feature presence patterns for subsequent local training iterations and personalized inference operations.

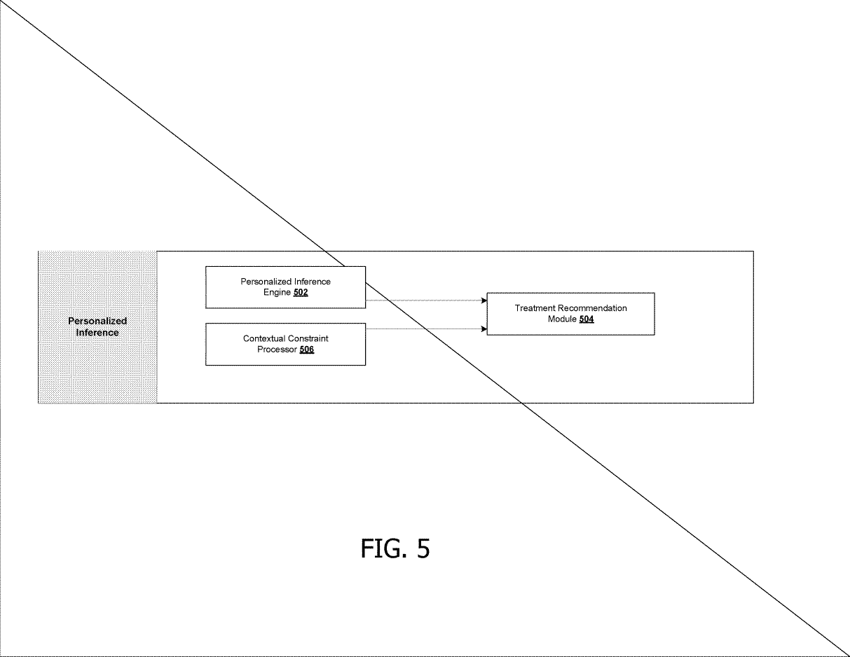

Referring to FIG. 5, the personalized inference and treatment recommendation pipeline operates at each local survival modeling node 102 to generate patient-specific treatment recommendations. The personalized inference and treatment recommendation pipeline provides treatment recommendations that combines globally learned parameters with local contextual constraints. In this way, the local survival modeling node 102 provides institution-specific or patient specific recommendations while benefiting from the collaborative training generated through the federated learning system 100. The local survival modeling node 102 receives patient data for a patient from the local patient data repository 108 when a clinician initiates a treatment recommendation request for the patient. The patient data comprises at least one of electronic health records, clinical registry data, genomic data, or imaging-derived features stored at the healthcare institution. The embedding engine 204 transforms the patient data into embedded feature representations using the same transformation process described above with reference to the local model training process 400.

With continued reference to FIG. 5, a personalized inference engine 502 at the local survival modeling node 102 generates a patient-specific survival prediction by integrating the updated global model parameters generated according to the local model training process 400, described above, with the embedded feature representations. The personalized inference engine 502 applies the aggregated hazard coefficients from the updated global model parameters to the embedded feature representations for the patient to compute a predicted risk score. The patient-specific survival prediction comprises a survival probability estimate for the patient that characterizes the probability of the patient surviving to specified time horizons based on the patient's clinical characteristics and the knowledge aggregated from participating healthcare institutions.

The personalized inference engine 502 generates patient-specific predictions by combining global parameters, local parameters, patient embeddings, and contextual factors using a function g expressed as:

$${I}_{k} = g ( {θ}_{global} , {θ}_{local} , {E}_{k} , {C}_{k} )$$

where Iₖ represents the personalized inference for patient k, θ_{global} represents the updated global model parameters received from the federated learning server 110, θ_{local} represents the local model update parameters trained on institution-specific patient data, Eₖ represents the embedded feature representations for patient k, and Cₖ represents contextual factors for patient k. This dual-level inference approach enables the personalized inference engine 502 to leverage both the broad population-level knowledge encoded in the updated global model parameters and the institution-specific patterns captured in the local model update parameters. Generating the patient-specific survival prediction further comprises integrating local model update parameters trained on institution-specific patient data with the updated global model parameters, enabling the personalized inference engine 502 to account for local patient population characteristics that differ from the aggregated nationwide patterns.

As further shown in FIG. 5, a contextual constraint processor 506 processes local contextual factors for the patient to inform the treatment recommendation process. The local contextual factors comprise at least one of cost constraints, insurance coverage, or treatment availability at the healthcare institution. For example, the contextual constraint processor 506 may retrieve information about the patient's insurance coverage and determines which treatments are covered under the patient's insurance plan. Alternatively, or additionally the contextual constraint processor 506 may retrieve information about treatment costs and compares treatment costs against the patient's financial constraints or institutional cost guidelines. In another example, the contextual constraint processor 506 may retrieve information about treatment availability at the healthcare institution, including availability of specific medications, availability of specialized equipment, and availability of clinical staff with expertise in specific treatment modalities. The contextual constraint processor 506 encodes the local contextual factors into a structured representation that the treatment recommendation module 504 processes alongside the patient-specific survival prediction.

With continued reference to FIG. 5, a treatment recommendation module 504 generates ranked treatment options for the patient based on the patient-specific survival prediction and the local contextual factors processed by the contextual constraint processor 506. The treatment recommendation module 504 receives inputs from both the personalized inference engine 502 and the contextual constraint processor 506 to evaluate multiple candidate treatment options and compute a ranking score for each treatment option based on multiple criteria. Generating ranked treatment options comprises ranking treatment options based on at least one of predicted survival probability, side-effect risk, or expected treatment efficacy.

The treatment recommendation module 504 computes predicted survival probability for each treatment option by applying the personalized inference engine 502 to generate survival predictions under each candidate treatment scenario. The treatment recommendation module 504 retrieves side-effect risk information for each treatment option from clinical databases or knowledge bases that characterize adverse event profiles for different treatments. The treatment recommendation module 504 retrieves expected treatment efficacy information based on clinical trial data, real-world evidence, or institution-specific outcome data. The treatment recommendation module 504 combines the predicted survival probability, side-effect risk, expected treatment efficacy, and local contextual factors to compute a composite ranking score for each treatment option. The treatment recommendation module 504 sorts the treatment options by the composite ranking score and presents the ranked treatment options to the clinician for review and decision-making.

The federated survival analysis system supports a broad range of healthcare applications that benefit from collaborative model training across distributed institutions while preserving patient privacy. The federated survival analysis system may be applied to chronic disease modeling, rare disease collaboration, pharmaceutical research, and population health analytics. In precision oncology applications, the federated learning system enables oncology centers to collaboratively train survival models on genomic and clinical data without sharing patient-level genomic information across institutional boundaries. The federated architecture also extends to chronic disease management for conditions such as diabetes and heart disease, where long-term tracking of disease progression across diverse patient populations improves predictive accuracy. Healthcare institutions collaboratively train survival models that predict disease progression and outcomes, with the aggregated global model parameters capturing relationships between clinical measurements, medication regimens, and long-term outcomes learned from patients managed at diverse healthcare settings. For rare disease collaboration, where patient data is scarce and dispersed across specialized centers, the federated survival analysis system addresses the underlying challenge of limited sample sizes at individual institutions. The system enables rare disease specialists to pool knowledge from patients across multiple institutions without centralizing patient records, improving diagnostic accuracy and treatment protocol development for conditions that are challenging to study in isolated settings.

Pharmaceutical research applications, including drug discovery analytics, benefit from the federated architecture's ability to analyze molecular structures, biological pathways, and clinical trial results across multiple research institutions and pharmaceutical companies without compromising sensitive proprietary data. Pharmaceutical researchers analyze survival outcomes and treatment responses across multiple healthcare institutions and clinical trial sites without accessing patient-level data from each site, with the aggregated global model parameters encoding relationships between drug exposures, patient characteristics, and survival outcomes.

The federated survival analysis system also supports public health applications. In population health management, public health agencies and healthcare systems collaboratively analyze survival outcomes and risk factors across geographic regions and demographic populations without centralizing patient records from multiple institutions. During pandemic response scenarios, the system enables faster identification of effective treatments, prediction of outbreak hotspots, and enhanced resource allocation by aggregating patient data from healthcare systems without the delays and governance barriers associated with centralized data aggregation. Healthcare resource optimization applications leverage the global model parameter store for predictive hospital resource allocation using survival-adjusted patient flow forecasting. Healthcare systems collaboratively train predictive models that forecast patient flow and resource requirements based on survival-adjusted patient trajectories, enabling optimization of staffing, equipment utilization, and supply chain management across healthcare networks.

In personalized mental health applications, mental health providers and research institutions collaboratively train models that predict treatment response and long-term outcomes for patients with conditions including depression and anxiety. The system analyzes behavioral patterns, treatment responses, and neuroimaging data, with the aggregated global model parameters encoding relationships between patient characteristics, treatment modalities, and outcomes learned from patients treated at diverse mental health settings. Additional applications include clinical trial matching systems that identify eligible patients based on survival predictions and clinical characteristics, and insurance risk stratification applications that predict healthcare costs and outcomes for patient populations without sharing patient-level claims data across organizational boundaries.

Embodiments of the present disclosure may comprise a special purpose computer including a variety of computer hardware, as described in greater detail herein.

For purposes of illustration, programs and other executable program components may be shown as discrete blocks. It is recognized, however, that such programs and components reside at various times in different storage components of a computing device, and are executed by a data processor(s) of the device.

Although described in connection with an example computing system environment, the disclosed embodiments are operational with other special purpose computing system environments or configurations. The computing system environment is not intended to suggest any limitation as to the scope of use or functionality of any aspect of any disclosed or suggested embodiments. Moreover, the computing system environment should not be interpreted as having any dependency or requirement relating to any one or combination of components illustrated in the example operating environment. Examples of computing systems, environments, and/or configurations that may be suitable for use with one or more of the various disclosed embodiments include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the aspects of the present disclosure may be described in the general context of data and/or processor-executable instructions, such as program modules, stored one or more tangible, non-transitory storage media and executed by one or more processors or other devices. Generally, program modules include, but are not limited to, routines, programs, objects, components, and data structures that perform particular tasks or implement particular abstract data types. Aspects of the present disclosure may also be practiced in distributed computing environments where tasks are performed by remote processing devices that are linked through a communications network. In a distributed computing environment, program modules may be located in both local and remote storage media including memory storage devices.

In operation, processors, computers, and/or servers may execute the processor-executable instructions (e.g., software, firmware, and/or hardware) such as those illustrated herein to implement at least some disclosed embodiments.

Embodiments may be implemented with processor-executable instructions. The processor-executable instructions may be organized into one or more processor-executable components or modules on a tangible processor readable storage medium. Also, embodiments may be implemented with any number and organization of such components or modules. For example, aspects of the present disclosure are not limited to the specific processor-executable instructions or the specific components or modules illustrated in the figures and described herein. Other embodiments may include different processor-executable instructions or components having more or less functionality than illustrated and described herein.

The order of execution or performance of the operations in accordance with aspects of the present disclosure illustrated and described herein is not essential, unless otherwise specified. That is, the operations may be performed in any order, unless otherwise specified, and embodiments may include additional or fewer operations than those disclosed herein. For example, it is contemplated that executing or performing a particular operation before, contemporaneously with, or after another operation is within the scope at least some embodiments disclosed herein.

When introducing elements of embodiments and arrangements discussed herein, the articles “a,” “an,” “the,” and “said” are intended to mean that there are one or more of the elements. The terms “comprising,” “including,” and “having” are intended to be inclusive and mean that there may be additional elements other than the listed elements.

Not all of the depicted components illustrated or described may be required. In addition, some implementations and embodiments may include additional components. Variations in the arrangement and type of the components may be made without departing from the spirit or scope of the claims as set forth herein. Additional, different, or fewer components may be provided and components may be combined. Alternatively, or in addition, a component may be implemented by several components.

The above description illustrates embodiments by way of example and not by way of limitation. This description enables one skilled in the art to make and use at least some example arrangements and describes several embodiments, adaptations, variations, alternatives, and uses of the aspects of the disclosed arrangements, including what is presently believed to be the best mode of carrying out of at least some embodiments. Additionally, it is to be understood that the embodiments discussed and/or suggested herein are not limited in their application to the details of construction and the arrangement of components set forth in the following description or illustrated in the drawings. The arrangements described herein are capable of other embodiments and of being practiced or carried out in various ways. Also, it will be understood that the phraseology and terminology used herein is for the purpose of description and should not be regarded as limiting.

It will be apparent that modifications and variations are possible without departing from the scope defined in the appended claims. As various changes could be made in the above constructions and methods without departing from the scope of the embodiments discussed herein, it is intended that all matter contained in the above description and shown in the accompanying drawings shall be interpreted as illustrative and not in a limiting sense.

In view of the above, it will be seen that several advantages of disclosed embodiments are achieved and other advantageous results attained.

The Abstract and Summary are provided to help the reader quickly ascertain the nature of the technical disclosure. They are submitted with the understanding that they will not be used to interpret or limit the scope or meaning of the claims. The Summary is provided to introduce a selection of concepts in simplified form that are further described in the Detailed Description. The Summary is not intended to identify key features or essential features of the claimed subject matter, nor is it intended to be used as an aid in determining the claimed subject matter.

1. A method for updating a federated survival model across a plurality of healthcare institutions, the method comprising:
receiving, at a federated learning server, privacy-preserved model parameters from a plurality of local survival modeling nodes, wherein each local survival modeling node is deployed at a respective healthcare institution and is configured to:
train a local survival model to generate local model update parameters using embedded feature representations of institution-specific patient data and local outcome data,
apply differential privacy noise to the local model update parameters to generate the privacy-preserved model parameters, and
transmit the privacy-preserved model parameters to the federated learning server;
generating, by the federated learning server, feature availability vectors based on feature presence at each healthcare institution;
clustering, by the federated learning server, the plurality of healthcare institutions into groups based on similarity of feature presence patterns;
computing, within each cluster, component-wise weighted parameter averages using weights derived from institutional sample size and disease-severity metrics to generate updated global model parameters; and
distributing the updated global model parameters to the plurality of local survival modeling nodes.

2. The method of claim 1, wherein the local survival model comprises a Cox proportional hazards model.

3. The method of claim 1, wherein clustering the plurality of healthcare institutions into groups comprises applying KMeans clustering to the feature presence patterns.

4. The method of claim 1, wherein computing component-wise weighted parameter averages comprises aggregating parameters corresponding to shared features within each cluster.

5. The method of claim 1, wherein transmitting the privacy-preserved model parameters is further responsive to at least one of: availability of new patient outcome data, completion of a local training cycle, a periodic synchronization interval, or detection of a change in data distribution at a healthcare institution.

6. The method of claim 1, wherein each local survival modeling node is further configured to evaluate, before transmitting the privacy-preserved model parameters, model performance comparing a survival analysis metric to a predefined performance threshold.

7. The method of claim 6, wherein the survival analysis metric comprises a concordance index, and wherein the predefined performance threshold comprises a concordance index increase exceeding a predefined margin.

8. A method for generating personalized treatment recommendations using a federated survival model, the method comprising:
receiving, at a local survival modeling node deployed at a healthcare institution, updated global model parameters from a federated learning server, wherein the updated global model parameters are generated by:
clustering a plurality of healthcare institutions into groups based on feature presence patterns, and
computing, within each cluster, component-wise weighted parameter averages using weights derived from institutional sample size and disease-severity metrics;
receiving, at the local survival modeling node, patient data for a patient;
transforming the patient data into embedded feature representations;
generating, by a personalized inference engine at the local survival modeling node, a patient-specific survival prediction by integrating the updated global model parameters with the embedded feature representations;
processing, by a contextual constraint processor, local contextual factors for the patient, the local contextual factors comprising at least one of cost constraints, insurance coverage, or treatment availability; and
generating, by a treatment recommendation module, ranked treatment options for the patient based on the patient-specific survival prediction and the local contextual factors.

9. The method of claim 8, wherein generating ranked treatment options comprises ranking treatment options based on at least one of predicted survival probability, side-effect risk, or expected treatment efficacy.

10. The method of claim 8, wherein generating the patient-specific survival prediction further comprises integrating local model update parameters trained on institution-specific patient data with the updated global model parameters.

11. The method of claim 8, wherein the patient data comprises at least one of electronic health records, clinical registry data, genomic data, or imaging-derived features.

12. The method of claim 8, wherein the patient-specific survival prediction comprises a survival probability estimate for the patient.

13. The method of claim 8, wherein the patient data remains stored at the healthcare institution throughout transforming the patient data, generating the patient-specific survival prediction, and generating the ranked treatment options.

14. A decentralized federated learning system, comprising:
a plurality of local survival modeling nodes, each local survival modeling node deployed at a respective healthcare institution and comprising:
a local survival processor; and
a local survival memory coupled to the local survival processor, the local survival memory storing processor-executable instructions that, when executed, configure the local survival processor for:
training a local survival model on institution-specific patient data;
computing local model update parameters based on local outcome data;
injecting a calibrated noise parameter into the local model update parameters to generate privacy-preserved model parameters; and
transmitting the privacy-preserved model parameters to a federated learning server;
the federated learning server comprising:
a federated learning processor; and
a federated learning memory to the federated learning processor, the federated learning memory storing processor-executable instructions that, when executed, configure the federated learning processor for:
receiving the privacy-preserved model parameters from the plurality of local survival modeling nodes;
generating feature availability vectors based on feature presence at each healthcare institution;
clustering the plurality of healthcare institutions into groups based on similarity of feature presence patterns;
executing a severity-weighted aggregation engine configured to compute, within each cluster, component-wise weighted parameter averages using weights derived from institutional sample size and disease-severity metrics to generate updated global model parameters; and
distributing the updated global model parameters to the plurality of local survival modeling nodes.

15. The decentralized federated learning system of claim 14, wherein the local survival memory stores processor-executable instructions that, when executed, further configure the local survival processor for evaluating model performance based applying on the local model update parameters using a survival analysis metric; and wherein transmitting the privacy-preserved model parameters occurs in response to the survival analysis metric exceeding a predefined performance threshold.

16. The decentralized federated learning system of claim 15, wherein the survival analysis metric comprises at least one of a concordance index, a log-likelihood improvement, or a predictive performance measure.

17. The decentralized federated learning system of claim 14, wherein the local survival memory stores processor-executable instructions that, when executed, further configure the local survival processor for:
executing an embedding engine configured to transform the institution-specific patient data into high-dimensional feature vectors prior to training the local survival model.

18. The decentralized federated learning system of claim 14, wherein the severity-weighted aggregation engine is configured to perform aggregation over shared feature subsets within each cluster.

19. The decentralized federated learning system of claim 14, wherein the local survival memory stores processor-executable instructions that, when executed, further configure the local survival processor for:
executing a personalized inference engine configured to generate patient-specific treatment recommendations by integrating the updated global model parameters with local contextual factors for a patient.

20. The decentralized federated learning system of claim 14, wherein the local survival memory stores processor-executable instructions that, when executed, further configure the local survival processor for:
applying, before transmitting the privacy-preserved model parameters, one or more security mechanisms to the privacy-preserved model parameters, the one or more security mechanisms comprising at least one of encryption or an access control policy.
